(19) United States
(12) Plant Patent Application Publication
(10) Pub. No.: US 2013/0254952 P1
(43) Pub. Date: Sep. 26, 2013

(54) Osteospermum plant named 'KLEOE11185'

(50) ;

(71) Applicants: Nils KLEMM, Stuttgart (DE); Sankalp BHOSALE, Stuttgart (DE)

(72) Inventors: Nils KLEMM, Stuttgart (DE); Sankalp BHOSALE, Stuttgart (DE)

(73) Assignee: KLEMM+SOHN GMBH & CO. KG, Stuttgart (DE)

(21) Appl. No.: 13/506,047

(22) Filed: Mar. 22, 2012

(51) Int. Cl. A01H 5/00

(52) U.S. Cl. PLT/360

(57) ABSTRACT

An osteospermum variety named ‘KLEOE11185’ particularly distinguished by dark red-purple disc florets, uniformly enlarged disc florets of the corolla, and very strong contrasting flower color, is disclosed.

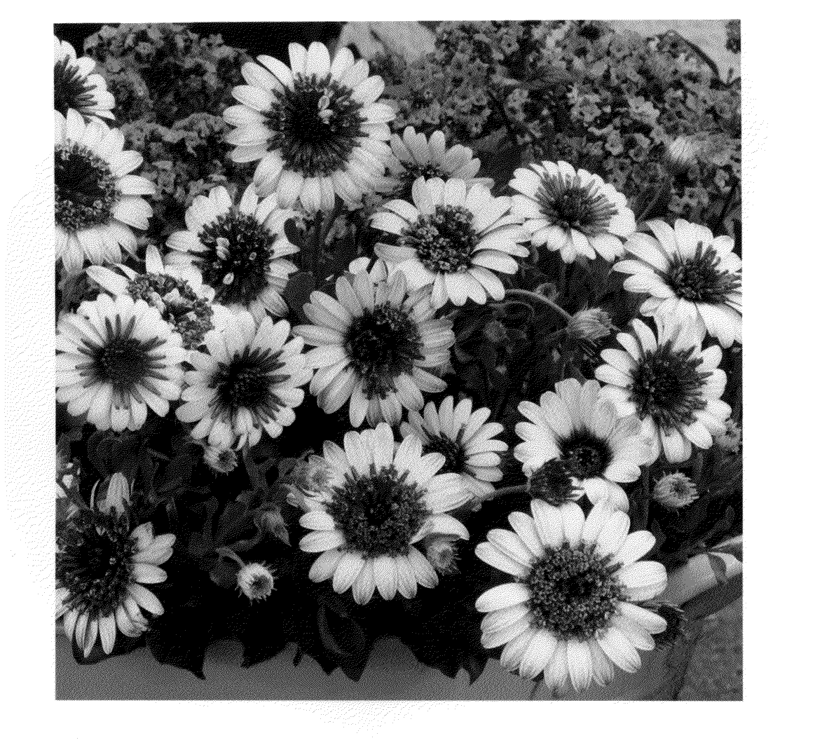

### GENUS AND SPECIES

Osteospermum ecklonis Norl.

### VARIETY DENOMINATION

‘KLEOE11185’

### BACKGROUND OF THE NEW PLANT

The present invention comprises a new and distinct variety of osteospermum, botanically known as Osteospermum ecklonis Norl., and hereinafter referred to by the variety name ‘KLEOE11185’. The new variety originated from a controlled cross conducted in the spring of 2007 in Nairobi, Kenya. The female parent was osteospermum plant named ‘KLEOE05116’ (U.S Plant Pat. No. 18,576), and the male parent was osteospermum plant named ‘Mutant 1’ (unpatented). A single plant selection was subsequently chosen for further evaluation and asexual propagation.

The new variety was first propagated via vegetative cuttings in May 2008 in Stuttgart, Germany and has been asexually reproduced repeatedly by vegetative cuttings for three to four generations. The present invention has been found to retain its distinctive characteristics through successive asexual propagations via vegetative cuttings.

Plant Breeder's Rights for this variety were applied for in the following countries: Switzerland on Jun. 28, 2011, the European Union on Jul. 7, 2011, Norway on Jul. 21, 2011 and Canada on Jan. 18, 2012. ‘KLEOE11185’ has not been made publicly available or sold more than one year prior to the filing date of this application.

### SUMMARY OF THE INVENTION

The following are the most outstanding and distinguishing characteristics of the new variety when grown under normal horticultural practices in Stuttgart, Germany.

1. Dark red-purple disc florets;

2. Uniformly enlarged disc florets of the corolla; and

3. Very strong contrasting flower color.

### DESCRIPTION OF THE PHOTOGRAPH

This new osteospermum plant is illustrated by the accompanying photograph; the colors shown are as true as can be reasonably obtained by conventional photographic procedures. The photograph was taken in spring 2011 on a 3-month old plant grown in a heated glass greenhouse in Stuttgart, Germany, under conditions which approximate those generally used in normal horticultural practice.

### DESCRIPTION OF THE NEW VARIETY

The following detailed descriptions set forth the distinctive characteristics of ‘KLEOE11185’. The data which define these characteristics were collected from asexual reproductions carried out in Stuttgart, Germany. The plant history was taken in the spring of 2011 on 6-month old plants grown in 12 centimeter pots. The plants were grown in a heated glass greenhouse in Stuttgart, Germany and pinched once at approximately 16 weeks old. Color readings were taken under natural and artificial light. Color references are primarily to the R.H.S. Colour Chart of The Royal Horticultural Society of London (R.H.S.), Fifth Edition (2007).

### DETAILED BOTANICAL DESCRIPTION OF THE NEW PLANT

Classification:
Family: Asteraceae
Botanical name: Osteospermum ecklonis Norl.
Common name: African daisy
Denomination: ‘KLEOE11185’
Parentage:
Female parent: Osteospermum plant named ‘KLEOE05116’ (U.S Plant Pat. No. 18,576)
Male parent: Osteospermum plant named ‘Mutant 1’ (unpatented)
Plant:
Form and habit: Herbaceous perennial, usually cultivated as an annual
Growth habit: Vigorous
Branching habit: Freely branching
Height (from top of soil): 29.0 cm
Width (including inflorescence): 19.0 cm
Propagation: Vegetative cuttings from tips
Time to produce a finished flowering plant: 14 to 16 weeks
Time to initiate and develop roots: 3 to 4 weeks
Root description: Freely rooting
Leaves:
Arrangement: Arranged in a whorl
Shape: Lanceolate
Apex: Acute
Base: Acute
Margin: Serrate
Color (both mature and immature leaves):
   Upper surface.13 RHS 137B.
   Lower surface.—RHS 137C.
Length: 6.5 cm
Width: 2.2 cm
Texture: Leathery
Petiole: Absent
Stems:
Total number of branches: Approximately 15
Length: 16.0 cm to 18.0 cm
Diameter: 0.5 cm
Internode length: 1.2 cm to 1.6 cm
Color: RHS 137C
Texture: Smooth
Inflorescence buds:
Shape: Obovate
Length: 1.1 cm
Diameter: 0.8 cm
Color (at tight bud just before the ray florets unfold): RHS 137A
Inflorescence:
Type: Single
Blooming habit: Spring to autumn
Quantity of inflorescences per plant: 8 to 10
Lastingness of the inflorescences on the plant: 10 to 14 days
Fragrance: Absent
Inflorescence diameter: 4.8 cm to 5.5 cm
Disc diameter: 3.0 cm to 3.2 cm
Disc florets:
Quantity per inflorescence: 40 to 60
Shape: Oblanceolate
Tube color:
   Closed.—RHS 70B.
   Mature.—RHS 70A.
Length: 1.2 cm to 1.5 cm
Diameter (at apex): 0.18 cm
Apex: Acute
Apex color: RHS N79B
Base: Fused to form a tube
Margin: Entire
Ray florets:
Quantity per inflorescence: 21
Shape: Oblanceolate
Color:
   Upper surface.—RHS 76D with apex and margins tinted RHS 71A.
   Lower surface.—RHS 70B.
Length: 2.3 cm to 2.6 cm
Width: 0.5 cm to 0.7 cm
Apex: Obtuse
Base: Acute
Margin: Entire
Texture (both surfaces): Smooth
Peduncle:
Length: 8.0 cm to 14.0 cm
Diameter: 0.18 cm
Texture: Rough
Color: RHS 137D
Phyllaries:
Arrangement: Single
Observed quantity per inflorescence: Approximately 21
Shape: Lanceolate
Color:
   Upper surface.—RHS 137A.
   Lower surface.—RHS 137D.
Length: 1.1 cm
Width: 0.15 cm
Apex: Acute
Base: Obtuse
Margin: Entire
Texture: Rough
Reproductive organs:
Androecium:
   Location.—Base of the disc floret corolla.
   Stamens.—Quantity: 40 to 60 Shape: Filamentous Color: RHS N77A Filament length: 0.5 cm Filament diameter: 0.02 cm.
   Anther.—Shape: Elliptical Color: RHS N77A Length: 0.11 cm Diameter: 0.08 cm.
   Pollen.—Color: RHS N13A Amount: Sparse.
Gynoecium:
   Location.—Base of the ray florets; stigmas in disc florets are degenerated and non-functional.
   Pistils.—Number: 21 Length: 0.35 cm Diameter: 0.02 cm.
   Stigma.—Color: RHS N77A Shape: Bipartite Length: 0.15 cm Diameter: 0.02 cm.
   Style.—Color: RHS N77A Length: 0.2 cm Diameter: 0.15 mm Shape: Filamentous.
Fruit and seed set: Seed set was observed.
Disease and insect/pest resistance: No specific observations were made.

### Comparison with Parental Varieties

‘KLEOE11185’ differs from the female parental osteospermum plant ‘KLEOE05116’ (U.S. Plant Pat. No. 18,576) in that ‘KLEOE11185’ has uniformly enlarged disc florets of the corolla, whereas ‘KLEOE05116’ has normal size disc florets of the corolla.

‘KLEOE11185’ differs from the male parental osteospermum plant ‘Mutant 1’ (unpatented) in that ‘KLEOE11185’ has dark red-purple disc florets of the corolla that are uniformly enlarged, whereas ‘Mutant 1’ has pink-purple disc florets of the corolla that are partially enlarged.

We claim:

1. A new and distinct variety of osteospermum plant named ‘KLEOE11185’ as shown and described herein.
